(19) United States
(12) Patent Application Publication
(10) Pub. No.: US 2009/0270834 A1
(43) Pub. Date: Oct. 29, 2009

(54) DRUG DELIVERY DEVICE

(75) Inventors: Giovanni Nisato, Eindhoven (NL); Jen-Eric Jack Martijn Rubingh, Geldrop (NL); Johan Frederik Dijksman, Weert (NL); Henry Timmermans, Best (NL); Mark Theo Meuwese, Nuenen (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/438,176

(22) PCT Filed: Aug. 13, 2007
(86) PCT No.: PCT/IB07/53199

§ 371 Date: Feb. 20, 2009

(30) Foreign Application Priority Data
Aug. 21, 2006 (EP) 06119215.9

(51) Int. Cl. A61M 5/30; A61M 31/00

(52) U.S. Cl. 604/500; 604/68; 604/93.01

(57) ABSTRACT

The invention refers to an electrically actuated, needle-free injection device. The main field of application is drug delivery. The device is based on a piezoelectric actuator (11). The device enables controlled, continuous, tuneable drug delivery. The device enables painless injection, personalized and programmable dosage profiles. The device is designed, both to pierce the epidermis for trans-epidermal drug delivery and to deliver controlled amounts of fluid (transdermal, electronic pill and implantable drug delivery). This type of device enables personalized drug delivery and is an enabling component for closed-loop drug delivery systems.

While oral delivery is the standard for drug delivery, many drugs cannot be formulated in such a format. For example, the treatment of diabetes, genetic disorders, and novel cancer treatments are based on (poly)peptides, which are destroyed in the gastro-intestinal tract. For these drugs the preferred delivery is injection, and appropriate formulations need to be developed or tuned to optimize therapeutic effect, which can be highly dependent on the patient and can vary during time. With a conventional drug injection, there is an overshoot delivery rate at short time, an undershoot at longer times and only in a small intermediate state the target therapeutic rate is reached, which is known as the bolus effect. It would thus be advantageous to have multiple injections of smaller doses to reach the optimal delivery rate.

Transdermal drug delivery, i.e. drug delivery directly through the skin, is increasingly used for delivery of drugs. The top layer of the skin is the stratum corneum (SC), the main layer insuring barrier properties of the skin, which essentially consists of dead cells (corneocytes) surrounded by lipid bilayers. In particular, it is recognized that jet-based systems can be used for transdermal drug delivery. In these systems, the fluid to be dispensed is accelerated to speeds high enough to disrupt the stratum corneum and to penetrate in the epidermis and dermis, accessing peripheral blood vessels. Such jet systems are able to penetrate the multiple layers constituting the skin and deliver drugs into the micro-vascularization of the dermis (subdermal injection), thereby achieving systemic drug delivery. For example, patent application US20020045911 A1 teaches high speed injection based on gas propulsion induced by gas discharge. The disadvantages are that high voltages are required and that the possible repetition rate is very low, thus precluding most meaningful medical applications.

It is therefore an object of the present invention to provide a drug delivery device with improved personalised drug delivery.

The above objective is accomplished by a drug delivery device, comprising a casing with a fluid chamber, a membrane forming a wall of the fluid chamber, the fluid chamber further comprising at least one exit orifice and the membrane being piezoelectrically actuatable for fluid ejection from the fluid chamber through the exit orifice, wherein a speed of the fluid ejection is adjustable by controlling the piezoelectric actuation of the membrane. Particularly, the inventive device is an electrically driven needle less injection device, based on piezoelectric actuation. The person skilled in the art understands that the inventive device comprises an electric power supply of any suitable kind.

It is an advantage of the drug delivery device according to the invention, that it allows the delivery of accurately small amounts of the drug per injection. The drug delivery is thus, for example, controllable and tuneable to the individual necessities of the patient. It is uncomplicated in use and provides painless delivery according to programmable dosage profiles which increases the patients' compliance. The personalised dosage of drugs is of great importance, for example, for the treatment of degenerative diseases such as Parkinson's, where the therapeutic range is very small, largely variable for different patients and even time dependent for a given patient.

The person skilled in the art understands, that the speed of the fluid ejection may advantageously be set to any desired value, for example, depending on how deep into the patient's skin the fluid shall be delivered. The speed of the fluid ejection may as well be reduced below values at which the human skin is ruptured which advantageously allows ingestible or implantable devices.

Preferably, the speed of the fluid ejection is adjustable to a high speed regime and at least one dispensing regime. Advantageously, the inventive device can be used both to pierce the epidermis, for example for trans-dermal drug delivery and to deliver controlled amounts of drug. The fluid ejection speed in the high speed regime is thus preferably at least sufficient for injecting the fluid through at least an outer layer of the skin of a patient. The top layer of the skin is the stratum corneum (SC), the main layer insuring barrier properties of the skin. The fluid to be ejected is accelerated to an ejection speed high enough to disrupt the stratum corneum, to penetrate and diffuse in the epidermis and dermis, accessing peripheral blood vessels.

Preferably, the fluid ejection speed in the high-speed regime is controllable, particularly between 60 m/s and 200 m/s. The inventive device thus provides a broad range for utilisation. The fluid ejection speed of 60 m/s is a typical speed for damage of soft tissues of biological nature such as bacterial films. The most preferred fluid ejection speed in the high speed regime for needle less drug injection is about 120 m/s to 150 m/s.

In the high speed regime, a voltage for piezoelectric actuation of the membrane is preferably stepwisely changeable, more preferably comprising a peak value between 20 V and 100 V and pulse duration between 10 μs and 1000 μs. In particular, a steep voltage rise, followed by a slow voltage decay is preferred, in order to eject fluid at maximum speed. The relatively high voltages advantageously create large volume displacements in the fluid chamber and thus fluid ejection volumes which particularly range between 1 nl to 8 nl (nano-litre).

The dispensing regime is particularly useful for controlled drug delivery. The voltage for driving the piezoelectric element is lower than for the high speed regime, thus advantageously saving electrical power and prolonging the service time of the electrically driven device, in particular if a battery is used as power supply. In a preferred embodiment, the dispensing regime is a jet-dispensing regime, the fluid ejection speed in particular being adjustable between 10 m/s and 60 m/s. The jet-dispensing regime is advantageously appropriate for un-clogging the nozzle of the inventive device from external contamination, further for dispensing fluids into biological material and reducing the possible damage to, for example, intestine lining, due to the relatively low speed of fluid ejection. This regime is advantageously adaptable to ingestible (so-called electronic pill) applications and controlled drug release in the intestine.

In a further preferred embodiment of the invention, the dispensing regime is a slow-dispensing regime, the fluid ejection speed particularly being adjustable between 0 m/s and 10 m/s. In this regime the device is operated as an advantageously highly reliable pump for accurately controlled small dosage of fluid, comparable to an ink-jet print head. This regime is most appropriate for accurate dosing of drugs for transdermal drug delivery subsequent to a modification such as piercing of the stratum corneum and underlying layers using the high speed regime of the inventive device. Further, the device in the slow-dispensing regime may be utilised for electronic pill applications and implantable pumps, for example after the surrounding area has been cleaned up by using the inventive device in either the high speed regime or the jet-dispensing regime as described above.

A voltage for piezoelectric actuation of the membrane is preferably changeable in the dispensing regimes, i.e. in both the jet-dispensing regime and the slow-dispensing regime, such as to form square pulses, the pulses more preferable comprising a pulse duration of 10 μs to 1000 μs. Advantageously repetition rates for ejecting fluid ranging from 1 Hz to 1000 Hz are enabled.

In a preferred embodiment, the drug delivery device further comprises an intake, the fluid chamber being self filling via the intake, by capillary force. Advantageously, no overpressure in a feeding channel for supplying the drug is necessary. More preferably, the device is provided with a standardised intake nozzle for reliable assembly with standard tubing (e.g. 1 mm internal diameter).

The drug delivery device preferably comprises a piezoelectric transducer, the membrane being actuated by the piezoelectric transducer. The piezoelectric transducer is in particular a multilayer ceramic. It is an advantage of this embodiment, that piezoelectric transducers are reliable and cheap. The inventive device may be mass manufactured at low production costs. For the sake of mass manufacturing, the piezoelectric transducer will usually be connected to the membrane, for example by gluing. In an alternative embodiment, the membrane is decoupled from the piezoelectric transducer, which allows a disassembly of the device for inspection, tuning and cleaning.

In a further preferred embodiment, the drug delivery device comprises a detection means for detecting the pressure in the fluid chamber, the detection means more preferable being a piezoelectric transducer. The person skilled in the art understands, that, most preferably, the membrane-actuating piezoelectric transducer is used additionally as detection means. The inventive device is thus advantageously equipped with a self-diagnosis feature. Upon application of voltage square pulses, the response function of the piezoelectric element changes drastically if the nozzle is clogged or if there is air in the nozzle chamber. Further, the inventive drug delivery device is advantageously provided with an injection detection feature. The fluid in the neighbourhood of the exit orifice determines the acoustics of the system. If a fluid ejection does not permeate the skin completely, the area in front of the exit orifice is wetted, which can be detected in the form of higher frequency components of the (time) Fourier transformed signal of the voltage signal from the piezoelectric unit after the square voltage pulse (or the step-wise voltage) is applied.

In an alternative embodiment, the membrane is an active piezoelectric membrane. The active piezoelectric membrane is itself a piezoelectric actuator by itself and does not need a relatively bulky, external piezoelectric transducer. Advantageously, the inventive device may be built smaller, in particular thinner, with an active piezoelectric membrane. This embodiment is compatible with semi-conductor based high volume manufacturing and provides a low cost alternative to multilayer piezoelectric ceramics.

In a further preferred embodiment, a voltage for piezoelectric actuation of the membrane is pulsed, the pulses being adjustable such that a frequency of the fluid ejection is multiplied by harmonic resonance in the fluid chamber. For example, the actual fluid ejection occurs both when the piezoelectrically actuated membrane expands and contracts the fluid chamber volume. In both cases, the movement of the membrane (either reducing or enlarging the fluid chamber) causes a pressure wave within the fluid in the chamber, resulting in an ejection of fluid. This leads to an advantageous frequency doubling of the ejected fluid with respect to the frequency of the applied square pulse voltage signal. This frequency doubling or multiplication is preferably controlled by changing the actual shape of the applied pulse.

Another object of the invention is a drug delivery system comprising a drug delivery device as described in here before, further comprising a microcontroller for controlling the fluid ejection speed, an amount of fluid ejected and/or a frequency of fluid ejection. By the use of a microcontroller in the inventive system, the features of the drug delivery device can be exploited optimally. The drug delivery devices may be of small scale and the microcontroller may be remote from the device.

A further object of the invention is an ingestible electronic pill drug delivery system comprising a drug delivery device according to the invention. A so-called electronic pill is an ingestible pharmaceutical form which actively dispenses a drug in the intestine. In particular, the drug delivery device is run in the dispensing regimes for electronic pill applications.

A further object of the invention is an implantable drug delivery system comprising a drug delivery device according to the invention. In this case the device is implanted, for example under the skin.

A further object of the invention is a needle less transdermal drug delivery system comprising a drug delivery device according to the invention. Advantageously the high speed regime is used for piercing the outer layer of the skin and at least one of the above described dispensing regimes is used for controlled delivery of drug through the ruptured skin.

A further object of the invention is a method for administration of a drug to a patient by a drug delivery system comprising a drug delivery device according to the invention, comprising the steps of
piercing at least an outer layer of the patient's skin by ejecting the fluid in a high speed regime at an ejection speed of more than 60 m/s,
dispensing the drug through the patient's outer skin layer by ejecting the fluid in a dispensing regime at an ejection speed below 60 m/s, preferably in a slow-dispensing regime below 10 m/s.

An advantage of the method is, that it takes several hours for the Stratum Corneum to close-up after being ruptured, so that the piercing in the high speed regime can be followed by a slower, gentler stream at lower speed for drug dispensing in the dispensing regime.

Preferably, the amount of ejected fluid and/or the frequency of fluid ejection, particularly in the dispensing regime, is controlled by a microcontroller, in particular depending upon a specific blood concentration of the drug. Advantageously, the continuous administration of appropriate drug formulations are tuned to optimise the therapeutic effect, depending upon the individual patient and, for example, the time of day.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

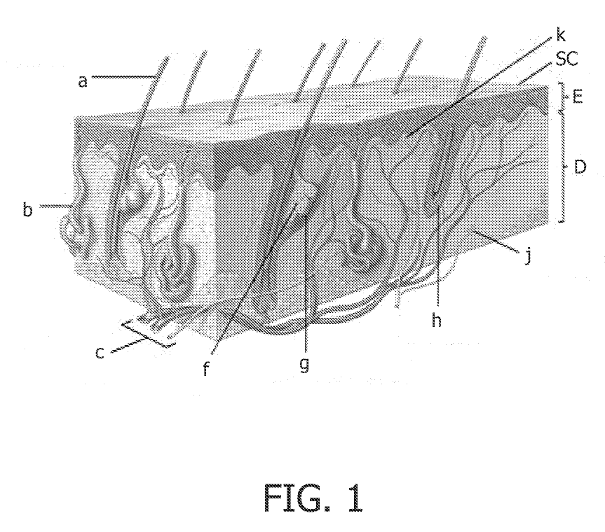

FIG. 1 illustrates the composition of human skin in a schematic cross section.

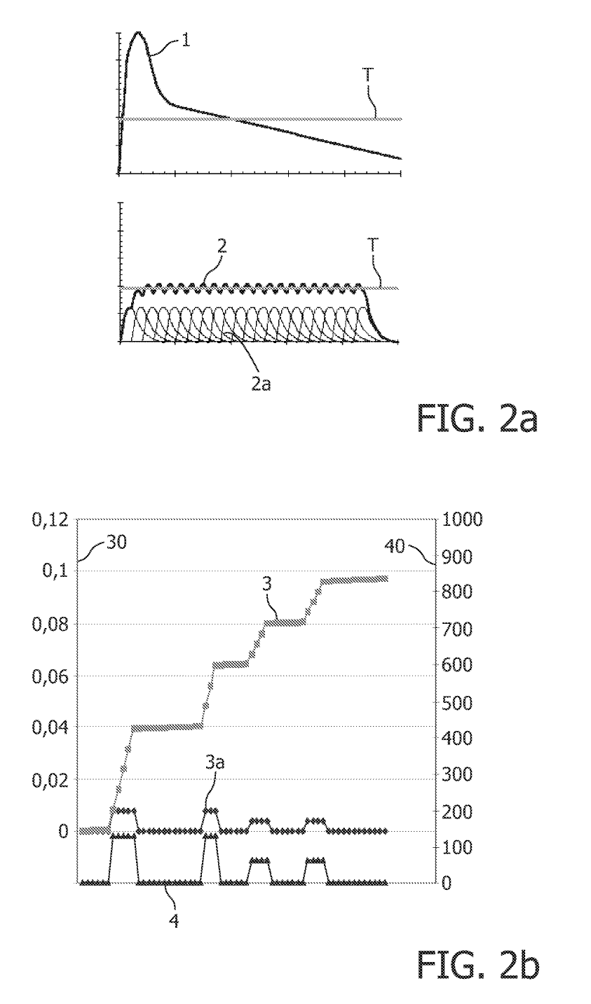

FIGS. 2a and 2b illustrate the release rate of the inventive drug delivery device versus conventional devices in diagrams.

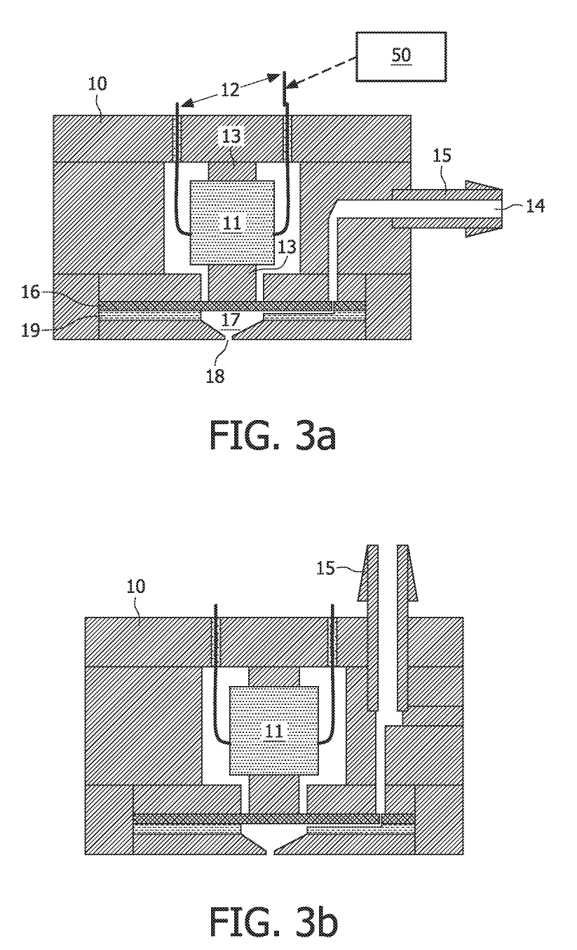

FIGS. 3a and 3b show schematically a first embodiment of the drug delivery device according to the present invention.

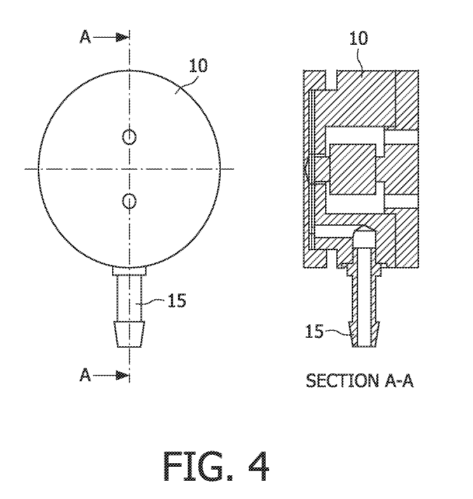

FIG. 4 shows a side elevation and a cross section of the embodiment of FIG. 3a.

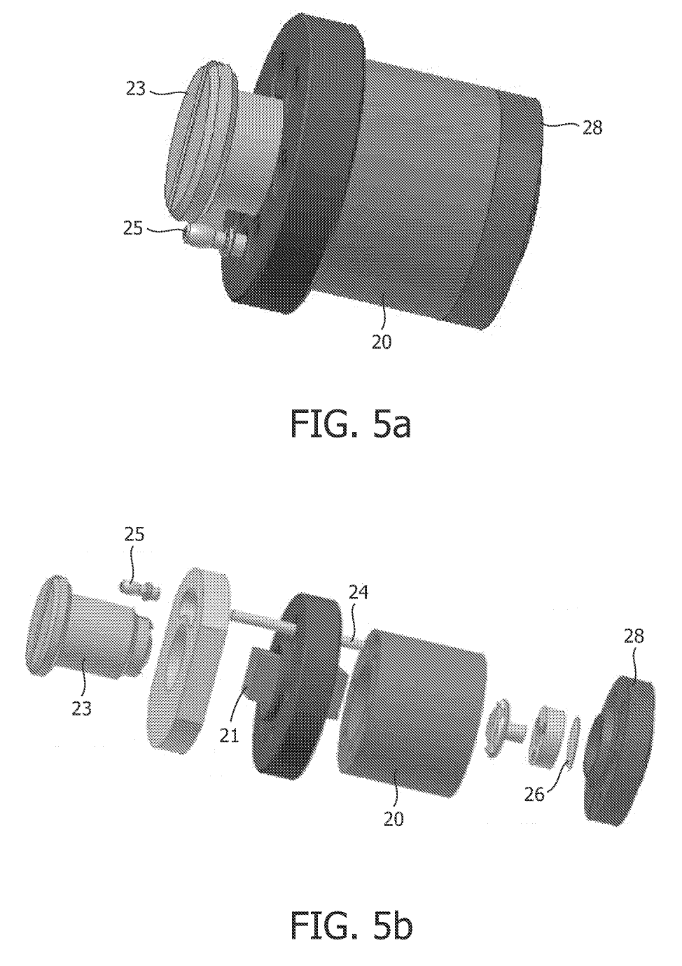

FIGS. 5a and 5b show schematically a second embodiment of the drug delivery device according to the present invention.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes.

Where an indefinite or definite article is used when referring to a singular noun, e.g. “a”, “an”, “the”, this includes a plural of that noun unless something else is specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

In FIG. 1, a schematic cross section of the human skin is depicted with hair shafts (a), sweat glands (b), a nerve and vascular supply (c), sebaceous glands (f), arrector pili muscles (g), hair follicles (h), Pacinian corpuscles ( ) and nerve endings (k). Transdermal drug delivery, i.e. drug delivery directly through the skin, is increasingly used for controlled and/or continuous delivery of drugs. Skin is an essential organ insuring both protection from external pathogens and preventing water loss. In both cases the barrier properties of skin, which are the result of millions of years of biological evolution, are essential to our survival. The top layer of the skin is the stratum corneum (SC), the main layer insuring barrier properties of the skin, which essentially consists of dead cells (corneocytes) surrounded by lipid bilayers. Due to their respective composition and structures, the stratum corneum is mostly hydrophobic and impermeable while the lower layers, epidermis (E) and dermis (D), are mostly hydrophilic. As a consequence, molecules with low molecular weight of less than 5 kilodalton (kDa) and with a lipophilic character tend to permeate the skin rather than large, hydrophilic molecules.

FIG. 2a shows two diagrams of a drug release rate over the time. The upper diagram indicates the development after conventional drug release in curve 1. In the lower diagram, curve 2 shows the overall rate development with a tunable drug delivery device according to the invention. The curves 2a represent the repeated short drug injections by the inventive drug delivery device. With the conventional drug delivery, there is an overshoot delivery rate 1 at short time, an undershoot at longer times and only in a small intermediate state the target therapeutic rate (T) is reached. By the multiple injection of smaller doses 2a the delivery rate 2 is constantly near the optimal delivery rate (T).

An example of personalized dosage of drugs is given in FIG. 2b, for example for the treatment of degenerative disease such as Parkinson's, where the therapeutic range is very small, largely variable for different patients and even time dependent for a given patient. In FIG. 2b, another diagram shows the injected dose 3a and total dose 3 in millilitres on the left axis of ordinates 30 over a time of about one hour. The injected dose 3a shows four injection periods. Curve 4 refers to the right axis of ordinates 40, representing an injection rate in nanolitres per second. It can be seen that the injection rate is higher in the first two injection periods than in the third and fourth injection period.

In FIGS. 3a and 3b, a first embodiment of the drug delivery device is schematically depicted, comprising of a casing 10, a piezoelectric transducer 11, mechanically coupled by a support structure 13 to the casing 10 at a first side and to a membrane 16 at the other side. The piezoelectric transducer 11, for example a small bulk piezoelectric transducer of multi layer ceramic is driven via power lines 12 which connect the piezoelectric transducer 11 to a driving unit (not shown). A microcontroller 50 controls the inventive device, in particular the supply of the piezoelectric transducer 11. The membrane 16 forms a wall of a fluid chamber 17 which comprises an outlet orifice 18 and which is connected to a fluid supply line 14. The fluid supply line 14 leads through the membrane 16 remote from the fluid chamber and runs at least partly between the membrane 16 and in interlayer 19. Fluid is supplied to the device via the intake connection 15 which is located at one side of the device. As depicted in FIG. 3b, the intake connection can as well be arranged at the upper side of the device, opposite to the outlet orifice 18.

During driving of the piezoelectric transducer 11, the piezoelectric transducer 11 expands and pushes on the flexible membrane 16. This compresses the fluid in the fluid chamber 17, resulting in a pressure build up and as a consequence a fluid flow out of the exit orifice 18. The exit orifice 18 is formed as a nozzle with a diameter typically ranging from 10 μm to 200 μm and a length between 50 μm to 200 μm. As soon as the driving of the piezoelectric transducer 11 stops, both the piezoelectric transducer 11 and the membrane 16 return to their rest states and fluid will enter the fluid chamber 17 through the fluid supply line 14 by capillary force. The fluid supply line 14 can be connected to a fluid reservoir (not shown) via the intake connection 15.

In order to generate a high-speed fluid ejection, the inventive device is mechanically stiff. If there was too much mechanical deformation of the device during driving of the piezoelectric transducer 11, the pressure in the fluid chamber would be too low to generate a high speed fluid ejection. Further, the relation between the length and diameter of the fluid supply line 14 and the length and diameter of the nozzle 18 determine the functioning of the inventive device.

The material of choice for the construction of the drug delivery device is stainless steel (if necessary coated with, for example, silver for medical compliance), but also other materials with the correct mechanical properties can be used such as: titanium, aluminum, ceramic, glass, bronze, brass. The device also needs to withstand sterilization procedures. The components are preferably assembled using two-component epoxy adhesives. The drug delivery device can be coated with, e.g. fluorinated polymers, to modify the contact angle and make the system amenable to non-water based solvents. This is of particular interest for drugs that are poorly soluble in water, but are soluble in less polar solvents.

The piezoelectric transducer 11 is driven using a voltage, which is applied to the piezoelectric transducer 11. In normal operation, the voltage can vary between 0 to 1000 Volts, most preferably between 0 and 100 V (or, using a multi-stack piezo-electric element with an electric field density of up to 1V/μm). Increasing the voltage increases the speed of the fluid ejection. The length of the voltage pulse normally varies between 10 μs and 1000 μs. In order to eject droplets at high fluid ejection speed, it is advantageous to use a special voltage pulse. First, the volume of the fluid chamber 17 has to be reduced stepwisely. Then, the pressure is released by the fact that the liquid starts to eject through the outlet orifice 18 or nozzle. As long as there is pressure, the fluid is accelerated through the nozzle. An opposing force is determined by the viscosity of the fluid. So, it depends on the magnitude of the pressure and the dimensions of the nozzle or outlet orifice 18 and the viscosity of the fluid, how long it takes until the pressure in the fluid chamber 17 is back to ambient pressure and what fluid ejection speed will be possible. Dosing at low speed requires a square voltage pulse. Increasing the pulse length will influence the volume of the ejected fluid and to a certain extent also the speed. By changing the repetition rate of the block pulse (frequency), the amount of ejected fluid per second can be changed. Common frequencies lie between 1 to 1000 Hz. The fluid chamber 17 is self filling, driven by the surface tension of the fluid, thereby avoiding the need to apply an over-pressure of the fluid reservoir (not depicted).

In FIG. 4, the embodiment of FIG. 3a is shown on the left side in a side elevation of the casing 10 with the intake connection 15. On the right side, a cross section through the casing 10 and the intake connection 15 along the line A-A is shown.

In FIG. 5a, a second embodiment of the drug delivery device is schematically depicted in an assembled state, whereas in FIG. 5b the device is disassembled and depicted in an schematic exploded view. This second embodiment of the inventive device is similar to the previous one in its principle of functioning and capabilities. The key points of the second embodiment are versatility and a more powerful ejection element (piezoelectric transducer 21). Contrary to the previous embodiment, where all the device components are permanently sealed, in this device all key components can be fully disassembled for maintenance, disinfection or tuning of the device. The key components that can be disassembled are:
nozzle plate 28, preferably of stainless steel with nozzle diameters of 10 μm to 200 μm,
membrane 26, preferably of polyamide, stainless steel, annealed spring steel,
holder 23 for the piezoelectric transducer 21, provided with a screw fitting that allows a precise positioning of the piezoelectric transducer 21 against the membrane 26,
fluid intake connection 25 and fluid supply line 24.
   Contrary to the state of the art, the piezoelectric transducer 21 is mechanically decoupled from the membrane 26 (not glued), which allows for a replacement of all parts. These parts are screwed into the casing 20, which is preferably made of stainless steel.

1. Drug delivery device, comprising a casing (10, 20) with a fluid chamber (17), a membrane (16, 26) forming a wall of the fluid chamber, the fluid chamber further comprising at least one exit orifice (18, 28) and the membrane being piezoelectrically actuatable for fluid ejection from the fluid chamber (17) through the exit orifice, wherein a speed of the fluid ejection is adjustable by controlling the piezoelectric actuation of the membrane (16, 26).

2. Drug delivery device according to claim 1, wherein the speed of the fluid ejection is adjustable to a high speed regime and at least one dispensing regime.

3. Drug delivery device according to claim 2, wherein the fluid ejection speed in the high speed regime is sufficient for injecting the fluid through at least an outer layer of the skin of a patient.

4. Drug delivery device according to claim 2, wherein the fluid ejection speed in the high speed regime is controllable, preferably between 60 m/s and 200 m/s.

5. Drug delivery device according to claim 2, wherein a voltage for piezoelectric actuation of the membrane is stepwisely changeable in the high speed regime, preferably comprising a voltage peak value between 20 V and 100 V and pulse duration between 10 μs and 1000 μs.

6. Drug delivery device according to claim 2, wherein the dispensing regime is a jet-dispensing regime, the fluid ejection speed preferably being adjustable between 10 m/s and 60 m/s.

7. Drug delivery device according to claim 2, wherein the dispensing regime is a slow-dispensing regime, the fluid ejection speed preferably being adjustable between 0 m/s and 10 m/s.

8. Drug delivery device according to claim 2, wherein a voltage for piezoelectric actuation of the membrane is changeable in the dispensing regimes, such as to form square pulses, the pulses preferably comprising a pulse duration of 10 μs to 1000 μs.

9. Drug delivery device according to claim 1, further comprising a fluid intake (15, 25), the fluid chamber (17) being self filling via the fluid intake, by capillary force.

10. Drug delivery device according to claim 1, comprising an piezoelectric transducer (11, 21), the membrane (16, 26) being actuated by the piezoelectric transducer.

11. Drug delivery device according to claim 10, the membrane (26) being decoupled from the piezoelectric transducer (21).

12. Drug delivery device according to claim 1, further comprising a detection means for detecting the pressure in the fluid chamber (17), the detection means preferably being a piezoelectric transducer.

13. Drug delivery device according to claim 1, the membrane being an active piezoelectric membrane.

14. Drug delivery device according to claim 1, wherein a voltage for piezoelectric actuation of the membrane (16, 26) is pulsed, the pulses being adjustable such that a frequency of the fluid ejection is multiplied by harmonic resonance in the fluid chamber (17).

15. Drug delivery system comprising a drug delivery device according to claim 1, further comprising a microcontroller (50) for controlling the fluid ejection speed, an amount of fluid ejected and/or a frequency of fluid ejection.

16. Ingestible electronic pill drug delivery system comprising a drug delivery device according to claim 1.

17. Implantable drug delivery system comprising a drug delivery device according to claim 1.

18. Needle less transdermal drug delivery system comprising a drug delivery device according to claim 1.

19. Method for administration of a drug to a patient by a drug delivery system comprising a drug delivery device according to claim 1, comprising the steps of
piercing at least an outer layer of the patient's skin by ejecting the fluid in a high speed regime at an ejection speed of more than 60 m/s,
dispensing the drug through the patient's outer skin layer by ejecting the fluid in a dispensing regime at an ejection speed below 60 m/s, preferably in a slow-dispensing regime below 10 m/s.

20. Method according to claim 19, wherein the amount of ejected fluid and/or the frequency of fluid ejection in the dispensing regime is controlled by a microcontroller (50), in particular depending upon a specific blood concentration of the drug.
